(19) United States
(12) Patent Application Publication
(10) Pub. No.: US 2023/0129983 A1
(43) Pub. Date: Apr. 27, 2023

(54) MICROBIOME TRANSPLANTATION FOR OSTEOARTHRITIS THERAPY

(71) Applicants: Oklahoma Medical Research Foundation, Oklahoma City, OK (US); The Board of Regents of the University of Oklahoma, Norman, OK (US)

(72) Inventor: Matlock Jeffries, Oklahoma City, OK (US)

(21) Appl. No.: 17/971,072

(22) Filed: Oct. 21, 2022

Related U.S. Application Data
(60) Provisional application No. 63/270,854, filed on Oct. 22, 2021.

(51) Int. Cl. A61K 35/741; A61P 19/02; A61P 19/08; G16H 10/40; G16H 20/00; C12Q 1/04; G16H 50/20

(52) U.S. Cl. CPC A61K 35/741; A61P 19/02; A61P 19/08; G16H 10/40; G16H 20/00; C12Q 1/04; G16H 50/20

(57) ABSTRACT

The present invention includes composition and methods for preventing, reducing or eliminating progression of osteoarthritis in a human patient comprising identifying a human patient in need of treatment for progression of osteoarthritis associated with an altered intestinal flora; and providing the human patient with a composition comprising at least one of: Actinobacteria actinomycetales; Actinobacteria coriobacteriia; Coriobacteriia coriobacteriales; Bacilli bacillales; Bacilli lactobacillales Clostridia clostridiales; one or more agents that increase an amount of the Actinobacteria actinomycetales; Actinobacteria coriobacteriia; Coriobacteriia coriobacteriales; Bacilli bacillales; Bacilli lactobacillales Clostridia clostridiales in the intestinal flora of the human patient; or one or more agents that decrease the amount of Bacteroidetes and Firmicutes clostridia bacteria in an intestinal flora of the human patient bacteria in an intestinal flora of the human patient to prevent, reduce or eliminate progression of osteoarthritis.

### CROSS-REFERENCE TO RELATED APPLICATIONS

This application is a non-provisional patent application of and claims priority to U.S. provisional patent application Ser. No. 63/270,854 filed on Oct. 22, 2021, the contents of which are incorporated by reference in its entirety.

### TECHNICAL FIELD OF THE INVENTION

The present invention relates in general to the field of treatments for osteoarthritis, and more particularly, to microbiome transplantation as a post-injury therapy to prevent the development of post-traumatic osteoarthritis.

### INCORPORATION-BY-REFERENCE OF MATERIALS FILED ON COMPACT DISC

None.

### BACKGROUND OF THE INVENTION

Without limiting the scope of the invention, its background is described in connection with osteoarthritis.

Osteoarthritis (OA) is the most common form of arthritis and occurs when the protective cartilage that cushions the ends of the bones wears down over time. OA currently affects around 32.5 million U.S. adults. It was reported in 2014 that post-traumatic arthritis causes about 12% of osteoarthritis of the hip, knee, and ankle, meaning that it affected about 5.6 million people in the United States.

Currently, there are no cures or treatments for OA. Current therapies are all designed around treating the symptoms of OA (i.e., pain and movement) rather than treating the underlying disease. There is a major need for approved disease modifying therapies.

What are needed are novel technologies for treating or preventing OA disease rather than symptom management. Also needed are novel diagnostics, compositions, and treatments that will prevent, reduce, or eliminate the progression of osteoarthritis.

### SUMMARY OF THE INVENTION

As embodied and broadly described herein, an aspect of the present disclosure relates to a method of preventing, reducing, or eliminating progression of osteoarthritis in a human patient comprising: identifying a human patient in need of treatment for progression of osteoarthritis associated with an altered intestinal flora; and providing the human patient with at least one of: a composition comprising at least one of: Actinobacteria actinomycetales; Actinobacteria coriobacteriia; Coriobacteriia coriobacteriales; Bacilli bacillales; Bacilli lactobacillales Clostridia clostridiales; one or more agents that increase an amount of the Actinobacteria actinomycetales; Actinobacteria coriobacteriia; Coriobacteriia coriobacteriales; Bacilli bacillales; Bacilli lactobacillales Clostridia clostridiales in the intestinal flora of the human patient; or one or more agents that decrease the amount of Bacteroidetes and Firmicutes clostridia bacteria in an intestinal flora of the human patient, wherein the composition at least one of prevents, reduces or eliminates progression of osteoarthritis. In one aspect, the human patient has early symptomatic osteoarthritis or a traumatic injury. In another aspect, the composition further comprises an amino acid mixture that promotes growth of the at least one of: Actinobacteria actinomycetales; Actinobacteria coriobacteriia; Coriobacteriia coriobacteriales; Bacilli bacillales; Bacilli lactobacillales Clostridia clostridiales bacteria; or a transplantation with the at least one of: Actinobacteria actinomycetales; Actinobacteria coriobacteriia; Coriobacteriia coriobacteriales; Bacilli bacillales; Bacilli lactobacillales Clostridia clostridiales increases microbiome-mediated cartilage regeneration. In another aspect, the human patient does not receive a concurrent antibiotic or a probiotic therapy. In another aspect, the method further comprises providing the human patient one or more bacteria selected from Actinobacteria actinomycetales; Actinobacteria coriobacteriia; Coriobacteriia coriobacteriales; Coriobacteriia Coriobacteriales coriobacteriaceae; Coriobacteriia coriobacteriales coriobacteriaceae adlercreutzia; Bacilli bacillales; Bacilli bacillales staphylococcaceae; Bacilli bacillales staphylococcaceae Staphylococcus; Bacilli lactobacillales aerococcaceae; Clostridia clostridiales dehalobacteriaceae; Clostridia clostridiales dehalobacteriaceae dehalobacterium. In another aspect, the method further comprises eliminating one or more of the bacterial selected from Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae. In another aspect, the method further comprises providing the human patient a bacterial composition that is modified to prevent, reduce or eliminate progression of osteoarthritis before, during, or after undergoing a colon cleanse treatment, delivered by colonoscope, wherein the bacterial composition is not a fecal transplant, or wherein bacteria are lab-grown, or wherein bacteria are lab-grown and customized to specifically modify a flora of a specific patient to prevent, reduce or eliminate progression of osteoarthritis.

As embodied and broadly described herein, an aspect of the present disclosure relates to a method of identifying a patient in need of preventing, reducing or eliminating progression of osteoarthritis that will benefit from a probiotic bacteria treatment comprising: identifying a human patient with progression of osteoarthritis; obtaining a biological sample from the patient that comprises gut intestinal flora; and determining whether the gut intestinal flora in the biological sample comprises a decrease in a presence of probiotic bacteria selected from at least one of: Actinobacteria actinomycetales; Actinobacteria coriobacteriia; Coriobacteriia coriobacteriales; Coriobacteriia Coriobacteriales coriobacteriaceae; Coriobacteriia coriobacteriales coriobacteriaceae adlercreutzia; Bacilli bacillales; Bacilli bacillales staphylococcaceae; Bacilli bacillales staphylococcaceae Staphylococcus; Bacilli lactobacillales aerococcaceae; Clostridia clostridiales dehalobacteriaceae; Clostridia clostridiales dehalobacteriaceae dehalobacterium; Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae, when compared to a human sample that does not have osteoarthritis. In one aspect, the method further comprises treating the patient with a composition comprising one or more probiotic bacteria selected from Actinobacteria actinomycetales; Actinobacteria coriobacteriia; Coriobacteriia coriobacteriales; Bacilli bacillales; Bacilli lactobacillales Clostridia clostridiales, wherein a composition comprises an amount effective to reduce or eliminate the progression of osteoarthritis; or a fecal transplant comprising at least one of: Actinobacteria actinomycetales; Actinobacteria coriobacteriia; Coriobacteriia coriobacteriales; Bacilli bacillales; Bacilli lactobacillales Clostridia clostridiales, or an amino acid mixture that increases the Actinobacteria actinomycetales; Actinobacteria coriobacteriia; Coriobacteriia coriobacteriales; Bacilli bacillales; Bacilli lactobacillales Clostridia clostridiales. In another aspect, the presence of a probiotic bacteria is determined by at least one of metagenomic shotgun sequencing of bacteria, quantitative PCR assays or metabolic LC-MS analysis of bacterial metabolites. In another aspect, the human patient has an injury that is chronic, mild, or undiagnosed that causes post-traumatic osteoarthritis. In another aspect, the patient did not receive a concurrent antibiotic or a probiotic therapy. In another aspect, the method further comprises eliminating one or more of the bacterial selected from Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae. In another aspect, the method further comprises providing the human patient a bacterial composition that is modified to correct a flora that will lead to osteoarthritis to a normal flora is provided before, during, or after undergoing a colon cleanse treatment, delivered by colonoscope, wherein the bacterial composition is not a fecal transplant, or wherein bacteria are lab-grown, or wherein bacteria are lab-grown and customized to specifically modify the flora of a specific patient for preventing, reducing or eliminating progression of osteoarthritis.

As embodied and broadly described herein, an aspect of the present disclosure relates to a composition for preventing, reducing or eliminating progression of osteoarthritis in a human patient comprising one or more probiotic bacteria selected from at least one of: Actinobacteria actinomycetales; Actinobacteria coriobacteriia; Coriobacteriia coriobacteriales; Bacilli bacillales; Bacilli lactobacillales Clostridia clostridiales, in an effective amount sufficient to prevent, reduce, or eliminate the progression of osteoarthritis. In another aspect, the composition is a fecal transplant, an amino acid mixture, or both to promotes growth of at least one of: Actinobacteria actinomycetales; Actinobacteria coriobacteriia; Coriobacteriia coriobacteriales; Bacilli bacillales; Bacilli lactobacillales Clostridia clostridiales; or an agent that eliminates one or more of the bacterial selected from Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae; or both.

As embodied and broadly described herein, an aspect of the present disclosure relates to an assay to determine a biological profile associated with a bacterial flora that exacerbates progression of osteoarthritis from a fecal sample from a patient that compares the biological profile from the patient to a database of a normal biological profile that does not have progression of osteoarthritis, wherein the bacterial flora detected is selected from at least one of: Actinobacteria actinomycetales; Actinobacteria coriobacteriia; Coriobacteriia coriobacteriales; Bacilli bacillales; Bacilli lactobacillales Clostridia clostridiales; Actinobacteria actinomycetales; Actinobacteria coriobacteriia; Coriobacteriia coriobacteriales; Bacilli bacillales; Bacilli lactobacillales Clostridia clostridiales; Bacteroidetes and Firmicutes clostridia bacteria in an intestinal flora of the human patient. In another aspect, the biological profile is further defined as comprising an array that specifically tests for an amino acid profile associated with a presence, absence, or amount of the at least one of: Actinobacteria actinomycetales; Actinobacteria coriobacteriia; Coriobacteriia coriobacteriales; Bacilli bacillales; Bacilli lactobacillales Clostridia clostridiales; Actinobacteria actinomycetales; Actinobacteria coriobacteriia; Coriobacteriia coriobacteriales; Bacilli bacillales; Bacilli lactobacillales Clostridia clostridiales; Bacteroidetes and Firmicutes clostridia bacteria; or wherein the biological profile is further defined as comprising an array that specifically tests for a nucleic acid profile associated with a presence, absence, or amount of the at least one of: Actinobacteria actinomycetales; Actinobacteria coriobacteriia; Coriobacteriia coriobacteriales; Bacilli bacillales; Bacilli lactobacillales Clostridia clostridiales; Actinobacteria actinomycetales; Actinobacteria coriobacteriia; Coriobacteriia coriobacteriales; Bacilli bacillales; Bacilli lactobacillales Clostridia clostridiales; Bacteroidetes and Firmicutes clostridia.

As embodied and broadly described herein, an aspect of the present disclosure relates to a kit for screening a patient for progression of osteoarthritis and choosing a treatment based on the screening, the kit comprising: a screening tool that screens the patient for a risk of progression of osteoarthritis; a fecal sample collection container for determining a bacterial flora in a fecal sample of the patient; instructions for implementing a treatment to prevent, reduce, or eliminate the progression of osteoarthritis; and a treatment composition comprising one or more probiotic bacteria selected from at least one of: Actinobacteria actinomycetales; Actinobacteria coriobacteriia; Coriobacteriia coriobacteriales; Bacilli bacillales; Bacilli lactobacillales Clostridia clostridiales bacteria in an intestinal flora of the patient, wherein the composition comprises an amount effective to prevent, reduce, or eliminate the progression of osteoarthritis. In another aspect, the bacterial flora detected is a presence or an amount of the at least one of: Actinobacteria actinomycetales; Actinobacteria coriobacteriia; Coriobacteriia coriobacteriales; Bacilli bacillales; Bacilli lactobacillales Clostridia clostridiales bacteria in a fecal sample. In another aspect, the kit comprises a container with reagents for detecting at a genus or species level the bacterial flora selected from at least one of Ion Torrent Personal Genome Machine (PGM), next-generation sequencing (NGS), or qualitative Polymerase Chain Reaction (qPCR).

As embodied and broadly described herein, an aspect of the present disclosure relates to a method of diagnosing and treating a patient for progression of osteoarthritis, the method comprising: using an algorithm implemented in a computer program to: screen a patient for progression of osteoarthritis risk by scoring a questionnaire based on answers from the patient to the questionnaire, the scoring providing a cumulative point total, and determining the progression of osteoarthritis of the patient based on the cumulative point total from the questionnaire; obtaining or having obtained a biological sample from the patient to determine the presence, absence, or amount of at least 1, 2, 3, 4, 5, 6, 7, 8, 9, 10, 11, 12, 13, 14, 15, 16, or 17 bacteria selected from: Actinobacteria actinomycetales; Actinobacteria coriobacteriia; Coriobacteriia coriobacteriales; Coriobacteriia Coriobacteriales coriobacteriaceae; Coriobacteriia coriobacteriales coriobacteriaceae adlercreutzia; Bacilli bacillales; Bacilli bacillales staphylococcaceae; Bacilli bacillales staphylococcaceae Staphylococcus; Bacilli lactobacillales aerococcaceae; Clostridia clostridiales dehalobacteriaceae; Clostridia clostridiales dehalobacteriaceae dehalobacterium; Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae in the biological sample; and choosing a progression of osteoarthritis treatment if the progression of osteoarthritis risk exceeds a threshold, wherein the treatment comprises: a composition comprising at least one of: Actinobacteria actinomycetales; Actinobacteria coriobacteriia; Coriobacteriia coriobacteriales; Bacilli bacillales; Bacilli lactobacillales Clostridia clostridiales; one or more agents that increase an amount of the Actinobacteria actinomycetales; Actinobacteria coriobacteriia; Coriobacteriia coriobacteriales; Bacilli bacillales; Bacilli lactobacillales Clostridia clostridiales in the intestinal flora of the human patient; or one or more agents that decrease the amount of Bacteroidetes and Firmicutes clostridia bacteria in an intestinal flora of the human patient, bacteria to the patient, wherein the composition or transplant comprises an amount effective of the bacteria to prevent, reduce, or eliminate the progression of osteoarthritis. In another aspect, the patient preparation instructions include following dietary modifications in accordance with the progression of osteoarthritis treatment to at least one of: increase the amount of at least one of: Actinobacteria actinomycetales; Actinobacteria coriobacteriia; Coriobacteriia coriobacteriales; Bacilli bacillales; Bacilli lactobacillales Clostridia clostridiales bacteria in the intestinal flora of the patient as being different from the patient with the progression of osteoarthritis when compared to a normal subject without osteoarthritis.

### BRIEF DESCRIPTION OF THE DRAWINGS

For a more complete understanding of the features and advantages of the present invention, reference is now made to the detailed description of the invention along with the accompanying figures and in which:

FIG. 1A is a graph that shows histologic OARSI scores 8 weeks after gut microbiome transplantation in adult B6 and MRL mice.

FIG. 1B is a graph that shows histologic OARSI scores 8 weeks after gut microbiome colonization of adult germ-free B6 animals with either MRL or B6 microbiota.

FIG. 1C is a graph that shows Gut transplantation effects on DMM histological outcomes.

FIG. 2 shows representative histologic sections for MRL-transplanted adult male C57BL/6J mice were also protected against PTOA development (mean summed OARSI score 2.5±0.7, p=0.009 vs. B6 vehicle, FIG. 1A/1B) and were indistinguishable from MRL vehicle mice (p=0.90).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It is shown herein that microbiome transplantation can be used as an immediate post-injury therapy to prevent the future development of post-traumatic osteoarthritis, and can be used in early symptomatic osteoarthritis to prevent future progression.

This is the first description of microbiome transplantation being utilized to reduce the severity of knee osteoarthritis in an animal model. There are currently no disease-modifying therapies available for OA treatment, which is the leading cause of chronic disability in the US. The present invention has several advantages, including: a single administration, high tolerability with minimal potential side effects, and is relatively inexpensive.

The probiotic bacteria, prebiotic agents, and/or xenobiotics for use with the present invention can be provided in a variety of dosage forms. For example, e.g., tablets, capsules, pills, powders, granules, elixirs, tinctures, suspensions, syrups, enemas, suppositories, and emulsions may be used to provide the probiotic bacteria, prebiotic agents, and/or xenobiotics of the present invention to a patient in need of therapy for brain injury associated fatigue and/or altered cognition.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference, and the like, relevant portions incorporated herein by reference.

For example, the probiotic bacteria, prebiotic agents, and/or xenobiotics may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with a non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

In one embodiment, gelatin capsules (gelcaps) may include the probiotic bacteria, prebiotic agents, and/or xenobiotics, and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the probiotic bacteria, prebiotic agents, and/or xenobiotics may be adapted for oral administration. Examples of suitable liquid dosage forms include liquids, gels, powders, tablets, gelcaps, solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, emulsifying agents, suspending agents, diluents, sweeteners, thickeners, and melting agents, mixtures thereof, and the like, that do not affect the viability of the probiotic bacteria or enhance the viability of the probiotic bacteria.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. Suitable pharmaceutical carriers are described in REMINGTON'S PHARMACEUTICAL SCIENCES, Mack Publishing Company, a standard reference text in this field, relevant portions incorporated herein by reference.

Gut microbiome transplantation from MRL/MpJ mice prevents post-traumatic osteoarthritis in C57BL6/J mice.

Purpose: MRL/MpJ mice are substantially protected from developing post-traumatic osteoarthritis (OA), a trait with strong correlation to the ability to heal ear wounds. The inventors have previously shown that this ear wound healing trait is partially determined by the gut microbiome, and that gut microbial transplantation into nonhealer mice can confer the ear wound healing phenotype. In this study, the inventors determined whether microbiome-mediated cartilage regeneration also applies to protection against post-traumatic osteoarthritis.

Methods: Twelve-week-old C57BL6/J mice were inoculated by oral gavage with diluted cecal contents from adult male MRL/MpJ mice (n=6). Separate groups of age-matched male C57BL6/J (n=5) and male MRL/MpJ mice (n=4) were gavaged with vehicle control. Destabilization of the medial meniscus (DMM) surgery was then performed unilaterally to induce OA. Eight weeks after DMM, mice were sacrificed, knee joints fixed in paraformaldehyde, decalcified, embedded in paraffin, stained with Safranin-O and histologically graded using the OARSI histopathology initiative recommendations by a blinded investigator. Differences in mean summed OARSI score per joint per group were determined by a Student t-test, p<0.05 was considered significant.

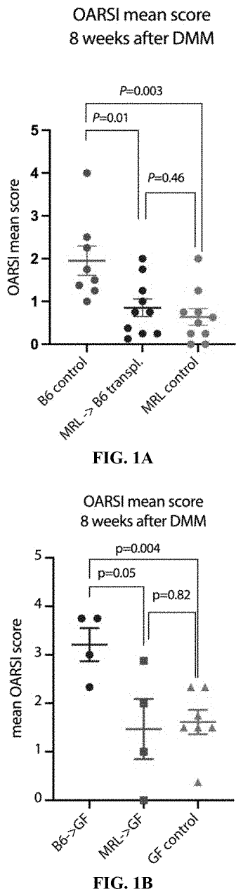

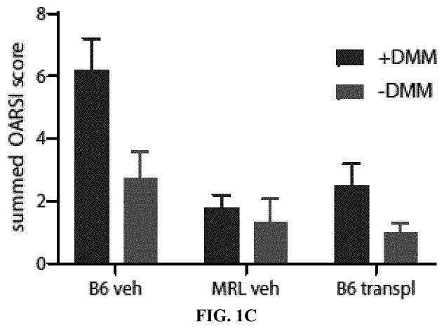

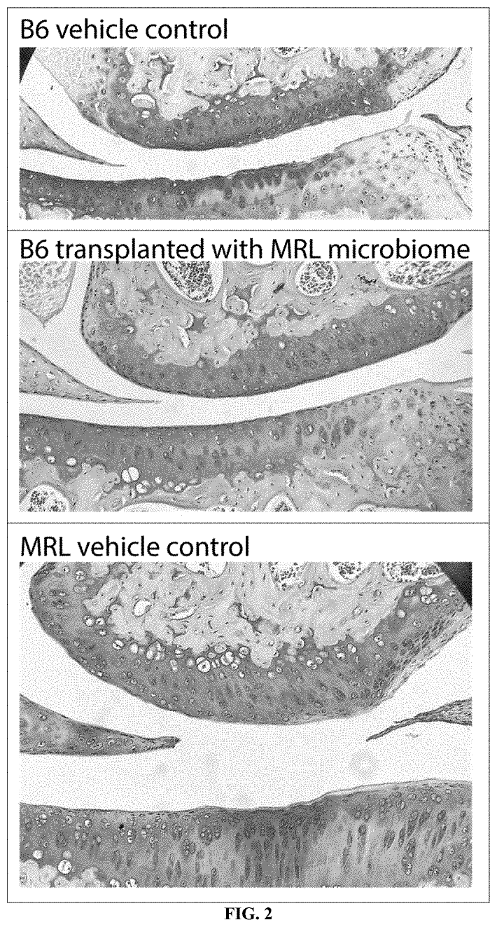

Results: Adult male MRL/MpJ were protected against post-traumatic OA when compared to adult male C57BL/6J mice. FIG. 1A is a graph that shows histologic OARSI scores 8 weeks after gut microbiome transplantation in adult B6 and MRL mice. FIG. 1B is a graph that shows histologic OARSI scores 8 weeks after gut microbiome colonization of adult germ-free B6 animals with either MRL or B6 microbiota. FIG. 1C is a graph that shows Gut transplantation effects on DMM histological outcomes. MRL-transplanted adult male C57BL/6J mice were also protected against PTOA development (mean summed OARSI score 2.5±0.7, p=0.009 vs. B6 vehicle, FIG. 1A) and were indistinguishable from MRL vehicle mice (p=0.90). Representative histologic sections are presented in FIG. 2.

Ethics Statement: The institutional review board and institutional animal care and use committees of all involved institutions approved this study; a detailed protocol was developed prior to beginning this study; this protocol and subsequent addenda were reviewed by the animal care and use committee of all involved institutions.

Mouse husbandry: Young male and female (4 week-old) C57BL6/J or MRL/MpJ mice were purchased from Jackson Laboratories (Bar Harbor, Me., USA) and housed at the Oklahoma Medical Research Foundation (OMRF). Breeding pairs were created and pups were used for some experiments. All animals were permitted ad libitum access to food and water (NIH31). The OMRF animal facility uses a 12-hour light-dark cycle. All animal husbandry procedures adhered to the NIH Guide for the Care and Use of Laboratory Animals. There were no adverse events (expected or unexpected) during the course of this experiment.

Mouse cecal microbiota transplantation procedure: Cecal donor mice (B6 or MRL, 10-14 weeks of age) were sacrificed and immediately dissected under sterile conditions. The cecum was removed and cecal contents transferred to a sterile tube containing a 1:1 mixture of glycerol and phosphate-buffered saline (5 mL), which was then filtered through a 100 μM filter. The resulting mixture was aliquoted and frozen at −80° C. for subsequent transplantation. At 3-4 weeks of age, recipient mice were pretreated with omeprazole (50 mg/kg body weight) as previously described [25,26]. Omeprazole was then sequentially administered via oral gavage once daily for 3 days prior to microbiome transfer. On the day of cecal transplantation, 100 μL, of transplant material (per 3-4-week-old recipient mouse) or 300 uL of transplant material (per 16-week-old adult mouse) was transplanted via oral gavage using flexible 30 mm polypropylene tubes (Instech, Plymouth Meeting, Pa., USA). Mice were then moved to clean cages and segregated by transplant group. For adult transplantation experiments, 12 week-old mice were given 50 mg/kg omeprazole once daily for 3 days, then transplanted on day 4, as above.

Mouse earhole puncture: At 6 weeks of age (for young animals) or 18 weeks of age (for adult animals), mice were ear punched using a 2 mm through-and-through ear punch.

Disruption of the medial meniscus (DMM) surgery: The procedure for inducing osteoarthritis in mice by destabilizing the medial meniscus was conducted as previously described(3). Briefly, mice were be anesthetized by isoflurane, placed on a warming pad, and the knee joint was prepped for aseptic surgery by shaving and washing the site with betadine or chlorhexidine. Instability of the knee joint was induced by transecting the anterior attachment of the medial meniscus to the tibial plateau. Then, a 3 mm longitudinal incision was created over the distal patella to proximal tibial plateau. The joint capsule immediately medial to the patellar tendon was incised with a #15 blade and the joint capsule opened with micro-iris scissors. Blunt dissection of the fat pad over the intercondylar area was performed to expose the intercondylar region, providing visualization of the meniscotibial ligament of the medial meniscus. The medial meniscotibial ligament was be transected with micro-surgical scissors, micro-surgical knife, or #11 blade, with the blade directed proximo-laterally, to induce destabilization of the medial meniscus (DMM). The joint capsule was then closed with a continuous 8-0 tapered Vicryl suture and the subcutaneous layer with 7-0 cutting Vicryl and the skin was closed by the application of tissue adhesive. Ketoprofen was administered perioperatively for pain relief. Left knee joints were left intact and are termed ‘left unoperated control joints’.

Mouse sacrifice procedures: Mice were sacrificed 4 weeks after ear punch (10 weeks of age for young and 22 weeks of age for adult animals). Whole blood was collected via cardiac puncture, allowed to clot for at least 30 minutes, then centrifuged and serum removed for subsequent analysis. Final earhole size was measured using digital calipers, investigators were blinded to mouse group during earhole size measurements. Cecal material was collected immediately after sacrificing animals and flash frozen in liquid nitrogen. Cecal DNA was extracted using a QIAamp DNA microbiome kit (Qiagen). Knees were resected en bloc, connective tissue and muscle cleaned from the joints, then the knees were fixed in 4% paraformaldehyde for 24 hours. Decalcification was performed with 10% EDTA, adjusted to pH 7.0-7.2, for 14 days, then progressive dehydration was performed with ethanol washes. Knee joints were submitted to tissue processing, where they were embedded in paraffin and sectioned onto slides, then stained with Safranin-O. Histopathologic grading was performed using the Osteoarthritis Research Society International (OARSI) grading system(4). Group differences were calculated using a two-tailed Student t-test.

16S ribosomal RNA (rRNA) gene sequencing: Microbial profiles were determined by sequencing a ˜460 bp region including the V3 and V4 variable region of bacterial 16S rRNA genes. The gene fragment was amplified from approximately 30 ng of DNA in each sample using a high-fidelity polymerase (NEB Q5, New England Biolabs)(5) and confirmed by 1% agarose gel electrophoresis. PCR master mixes were decontaminated with double-stranded DNAse treatment (PCR decontamination kit, Arcticzymes, Tromso, Norway). Sterile water was processed using the same procedure as a negative control. Illumina Nextera XT indices were attached (Illumina), pooled in equimolar amounts, and sequenced on an Illumina miSeq sequencer using a 300 bp paired-end sequencing protocol by the Clinical Genomics Center at OMRF.

TABLE 1
Histologic OARSI scores 8 weeks after gut microbiome
transplantation in adult B6 and MRL mice.

| | Mean ± standard | | |
|---|---|---|---|
| | error of the mean | P-value vs. | P-value vs. |
| | (SEM) OARSI score | B6 control | MRL control |
| B6 control | 1.95 ± 0.34 | n/a | 0.003 |
| (n = 8) | | | |
| MRL−>B6 | 0.85 ± 0.07 | 0.01 | 0.46 |
| transplant | | | |
| (n = 10) | | | |
| MRL control | 0.64 ± 0.19 | 0.003 | n/a |
| (n = 10) | | | |

Further studies were conducted following the finding of OA histological improvement in B6 (non-GF) animals following MRL microbiome transplantation. Germ-free animals were colonized at 12 weeks of age with cecal microbiome contents from either adult B6 or adult MRL animals, then underwent DMM surgery as in Table 1, with histological analysis 8 weeks later. Previous studies indicated that germ-free animals have reduced OA pathology compared to non-germ-free controls.

TABLE 2
Histologic OARSI scores 8 weeks after gut microbiome colonization
of adult germ-free B6 animals with either MRL or B6 microbiota.

| | Mean ± standard | P-value vs. | | |
|---|---|---|---|---|
| | error of the mean | B6-colonized | P-value vs. | P-value vs. |
| | (SEM) OARSI score | GF | MRL-colonized GF | GF control |
| B6-colonized | 3.03 ± 0.24 | n/a | 0.05 | 0.004 |
| GF (n = 4) | | | | |
| MRL-colonized | 1.47 ± 0.62 | 0.05 | n/a | 0.82 |
| GF (n = 4) | | | | |
| GF control | 1.33 ± 0.24 | 0.004 | 0.82 | n/a |
| (n = 5) | | | | |

16S rRNA OTU classification: Quality filtering, operational taxonomic unit (OTU) classification and microbial diversity analysis were performed using the Quantitative Insights into Microbial Ecology (QIIME) software package, version 1.9.1(6). Sequences were assigned to OTUs using the UCLUST algorithm(7) with a 97% pairwise identity threshold and taxonomy assigned using the GreenGenes 13_8 database(8).

Diversity analyses: Alpha diversity was characterized using the observed OTUs method following rarefaction to the lowest number of OTUs present per group (123,543). Beta diversity was evaluated on a variance-adjusted, weighted unifrac model. Principal component analysis was performed and an Adonis (permuted analysis of variance, a multi-factor PERMANOVA) test with 999 permutations was used to calculate the statistical significance of group differences(9,10).

Group analyses: Group analyses were performed using the linear discriminant analysis effect size (LEfSe) pipeline(11). LEfSe performs a non-parametric Kruskal-Wallis sum-rank test(12) to detect features with significant differential abundance between groups, P<0.01 was considered significant. Next, it uses a linear discriminant analysis (LDA)(13) to estimate the effect size of each differentially abundant feature. An LDA threshold of ≥2 was considered significant(14). QIIME was used to calculate group Benjamini-Hochberg FDR-corrected q-values; q≤0.01 was chosen as the ‘FDR-corrected’ significance threshold. For Gram status comparisons, differences were evaluated by Student t-tests, P<0.05 was considered statistically significant. Correlations were determined by comparing earhole closure rates of individual animals with microbiome clades, P<0.05 was considered statistically significant. No samples were excluded from analysis.

Prediction of metagenome content and imputed bacterial functional classification: The Phylogenetic Investigation of Communities by Reconstruction of Unobserved States (PICRUSt) software package(15) was used to impute bacterial metagenomes from 16S deep sequencing microbial DNA data, and functional annotation applied using the Kyoto Encyclopedia of Gene and Genomes (KEGG) catalog(16). Statistical analysis was performed using the Statistical Analysis of Metagenomic Profiles (STAMP) package(17). Statistical significance and effect sizes among the three groups (human OA-eroded, OA-intact, and control) were calculated using ANOVA. Statistical significance was defined as Benjamini-Hochberg FDR corrected P≤0.01.

TABLE 3
B6 transplant bacteria.

| | increased | LDA-effect | |
|---|---|---|---|
| clade | in | size | p-value |
| k_Bacteria._Actinobacteria.c_Actinobacteria._Actinomycetales.f_Microbacteriaceae | B6_transplant | 3.329885446 | 0.013874406 |
| k_Bacteria._Actinobacteria.c_Actinobacteria._Actinomycetales.f_Microbacteriaceae.g_{—} | B6_transplant | 3.256460232 | 0.013874406 |
| k_Bacteria._Firmicutes.c_Clostridia._Clostridiales.f_Dehalobacteriaceae.g_Dehalobacterium | B6_transplant | 3.063261889 | 0.020921335 |
| k_Bacteria._Firmicutes.c_Clostridia._Clostridiales.f_Dehalobacteriaceae | B6_transplant | 3.027102671 | 0.020921335 |
| k_Bacteria._Firmicutes.c_Bacilli._Lactobacillales.f_Aerococcaceae.g_{—} | B6_transplant | 2.815668124 | 0.013874406 |
| k_Bacteria._Firmicutes.c_Bacilli._Lactobacillales.f_Aerococcaceae | B6_transplant | 2.599510841 | 0.013874406 |

TABLE 4
Increase in MRL transplant

| | | LDA-effect | |
|---|---|---|---|
| clade | increased in | size | p-value |
| k_Bacteria._Firmicutes.c_Bacilli._Lactobacillales | MRL_vehicle | 4.25394042 | 0.020921 |
| k_Bacteria._Firmicutes.c_Bacilli._Lactobacillales.f_Lactobacillaceae | MRL_vehicle | 4.245536868 | 0.020921 |
| k_Bacteria._Firmicutes.c_Bacilli._Lactobacillales.f_Lactobacillaceae.g_Lactobacillus | MRL_vehicle | 4.231794854 | 0.020921 |
| k_Bacteria._Bacteroidetes.c_Bacteroidia._Bacteroidales.f_S24_7 | MRL_vehicle | 4.186056332 | 0.020921 |
| k_Bacteria._Bacteroidetes.c_Bacteroidia._Bacteroidales.f_S24_7 | MRL_vehicle | 4.167808158 | 0.020921 |
| k_Bacteria._Firmicutes.c_Bacilli._Lactobacillales.f_Aerococcaceae | MRL_vehicle | 3.601693772 | 0.047221 |
| k_Bacteria._Firmicutes.c_Bacilli._Lactobacillales.f_Aerococcaceae | MRL_vehicle | 3.54643039 | 0.047221 |
| k_Bacteria._Firmicutes.c_Clostridia._Clostridiales.f_Clostridiaceae.g_SMB53 | MRL_vehicle | 3.232976822 | 0.013874 |
| k_Bacteria._Firmicutes.c_Clostridia._Clostridiales.f_Peptostreptococcaceae | MRL_vehicle | 2.627048577 | 0.047221 |
| k_Bacteria._Firmicutes.c_Clostridia._Clostridiales.f_Peptostreptococcaceae | MRL_vehicle | 2.622581492 | 0.047221 |

Gut microbiome transplantation from OA-protected MRL mice into OA susceptible B6 mice reduces OA histopathology. These findings track those of improved earhole wound closure in transplanted B6 mice.

Table 5 shows the distinct microbiome composition is seen in B6 animals following MRL transplantation. The following microbiome clades are strongly associated with either OA protection (increased in transplant and MRL animals) or disease (decreased in transplant and MRL animals, increased in B6 animals).

TABLE 5
Microbiome clade OA Protection.

| Microbiome clade | Association | P value |
|---|---|---|
| k_Bacteria.p_Actinobacteria.c_Actinobacteria.o_Actinomycetales | OA protection | 0.01543 |
| k_Bacteria.p_Actinobacteria.c_Coriobacteriia | OA protection | 0.014306 |
| k_Bacteria.p_Actinobacteria.c_Coriobacteriia.o_Coriobacteriales | OA protection | 0.014306 |
| k_Bacteria.p_Actinobacteria.c_Coriobacteriia.o_Coriobacteriales.f_Coriobacteriaceae | OA protection | 0.014306 |
| k_Bacteria.p_Actinobacteria.c_Coriobacteriia.o_Coriobacteriales.f_Coriobacteriaceae.g_Adlercreutzia | OA protection | 0.014306 |
| k_Bacteria.p_Firmicutes.c_Bacilli.o_Bacillales | OA protection | 0.010198 |
| k_Bacteria.p_Firmicutes.c_Bacilli.o_Bacillales.f_Staphylococcaceae | OA protection | 0.010198 |
| k_Bacteria.p_Firmicutes.c_Bacilli.o_Bacillales.f_Staphylococcaceae.g_Staphylococcus | OA protection | 0.010198 |
| k_Bacteria.p_Firmicutes.c_Bacilli.o_Lactobacillales.f_Aerococcaceae | OA protection | 0.01543 |
| k_Bacteria.p_Firmicutes.c_Clostridia.o_Clostridiales.f_Dehalobacteriaceae | OA protection | 0.014306 |
| k_Bacteria.p_Firmicutes.c_Clostridia.o_Clostridiales.f_Dehalobacteriaceae.g_Dehalobacterium | MRL_vehicle | 0.014306 |

TABLE 6
Worsened OA Outcome.

| Microbiome clade | Association | P value |
|---|---|---|
| k_Bacteria.p_Bacteroidetes | Worsened | 0.014306 |
| | OA | |
| | outcome | |
| k_Bacteria.p_Bacteroidetes.c_Bacteroidia | Worsened | 0.014306 |
| | OA | |
| | outcome | |
| k_Bacteria.p_Bacteroidetes.c_Bacteroidia.o_Bacteroidales | Worsened | 0.014306 |
| | OA | |
| | outcome | |
| k_Bacteria.p_Bacteroidetes.c_Bacteroidia.o_Bacteroidales.f_Rikenellaceae | Worsened | 0.014085 |
| | OA | |
| | outcome | |
| k_Bacteria.p_Firmicutes.c_Clostridia.o_Clostridiales.f_Christensenellaceae | Worsened | 0.028254 |
| | OA | |
| | outcome | |
| k_Bacteria.p_Firmicutes.c_Clostridia.o_Clostridiales.f_Lachnospiraceae.g_Anaerostipes | Worsened | 0.013433 |
| | OA | |
| | outcome | |
| k_Bacteria.p_Firmicutes.c_Clostridia.o_Clostridiales.f_Ruminococcaceae | Worsened | 0.014306 |
| | OA | |
| | outcome | |

As embodied and broadly described herein, an aspect of the present disclosure relates to a method of preventing, reducing, or eliminating progression of osteoarthritis in a human patient comprising, consisting essentially of, or consisting of: identifying a human patient in need of treatment for progression of osteoarthritis associated with an altered intestinal flora; and providing the human patient with at least one of: a composition comprising at least one of: Actinobacteria actinomycetales; Actinobacteria coriobacteriia; Coriobacteriia coriobacteriales; Bacilli bacillales; Bacilli lactobacillales Clostridia clostridiales; one or more agents that increase an amount of the Actinobacteria actinomycetales; Actinobacteria coriobacteriia; Coriobacteriia coriobacteriales; Bacilli bacillales; Bacilli lactobacillales Clostridia clostridiales in the intestinal flora of the human patient; or one or more agents that decrease the amount of Bacteroidetes and Firmicutes clostridia bacteria in an intestinal flora of the human patient, wherein the composition at least one of prevents, reduces or eliminates progression of osteoarthritis. In one aspect, the human patient has early symptomatic osteoarthritis or a traumatic injury. In another aspect, the composition further comprises an amino acid mixture that promotes growth of the at least one of: Actinobacteria actinomycetales; Actinobacteria coriobacteriia; Coriobacteriia coriobacteriales; Bacilli bacillales; Bacilli lactobacillales Clostridia clostridiales bacteria; or a transplantation with the at least one of: Actinobacteria actinomycetales; Actinobacteria coriobacteriia; Coriobacteriia coriobacteriales; Bacilli bacillales; Bacilli lactobacillales Clostridia clostridiales increases microbiome-mediated cartilage regeneration. In another aspect, the human patient does not receive a concurrent antibiotic or a probiotic therapy. In another aspect, the method further comprises providing the human patient one or more bacteria selected from Actinobacteria actinomycetales; Actinobacteria coriobacteriia; Coriobacteriia coriobacteriales; Coriobacteriia Coriobacteriales coriobacteriaceae; Coriobacteriia coriobacteriales coriobacteriaceae adlercreutzia; Bacilli bacillales; Bacilli bacillales staphylococcaceae; Bacilli bacillales staphylococcaceae Staphylococcus; Bacilli lactobacillales aerococcaceae; Clostridia clostridiales dehalobacteriaceae; Clostridia clostridiales dehalobacteriaceae dehalobacterium. In another aspect, the method further comprises eliminating one or more of the bacterial selected from Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae. In another aspect, the method further comprises providing the human patient a bacterial composition that is modified to prevent, reduce or eliminate progression of osteoarthritis before, during, or after undergoing a colon cleanse treatment, delivered by colonoscope, wherein the bacterial composition is not a fecal transplant, or wherein bacteria are lab-grown, or wherein bacteria are lab-grown and customized to specifically modify a flora of a specific patient to prevent, reduce or eliminate progression of osteoarthritis.

As embodied and broadly described herein, an aspect of the present disclosure relates to a method of identifying a patient in need of preventing, reducing or eliminating progression of osteoarthritis that will benefit from a probiotic bacteria treatment comprising, consisting essentially of, or consisting of: identifying a human patient with progression of osteoarthritis; obtaining a biological sample from the patient that comprises gut intestinal flora; and determining whether the gut intestinal flora in the biological sample comprises a decrease in a presence of probiotic bacteria selected from at least one of: Actinobacteria actinomycetales; Actinobacteria coriobacteriia; Coriobacteriia coriobacteriales; Coriobacteriia Coriobacteriales coriobacteriaceae; Coriobacteriia coriobacteriales coriobacteriaceae adlercreutzia; Bacilli bacillales; Bacilli bacillales staphylococcaceae; Bacilli bacillales staphylococcaceae Staphylococcus; Bacilli lactobacillales aerococcaceae; Clostridia clostridiales dehalobacteriaceae; Clostridia clostridiales dehalobacteriaceae dehalobacterium; Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae, when compared to a human sample that does not have osteoarthritis. In one aspect, the method further comprises treating the patient with a composition comprising one or more probiotic bacteria selected from Actinobacteria actinomycetales; Actinobacteria coriobacteriia; Coriobacteriia coriobacteriales; Bacilli bacillales; Bacilli lactobacillales Clostridia clostridiales, wherein a composition comprises an amount effective to reduce or eliminate the progression of osteoarthritis; or a fecal transplant comprising at least one of: Actinobacteria actinomycetales; Actinobacteria coriobacteriia; Coriobacteriia coriobacteriales; Bacilli bacillales; Bacilli lactobacillales Clostridia clostridiales, or an amino acid mixture that increases the Actinobacteria actinomycetales; Actinobacteria coriobacteriia; Coriobacteriia coriobacteriales; Bacilli bacillales; Bacilli lactobacillales Clostridia clostridiales. In another aspect, the presence of a probiotic bacteria is determined by at least one of metagenomic shotgun sequencing of bacteria, quantitative PCR assays or metabolic LC-MS analysis of bacterial metabolites. In another aspect, the human patient has an injury that is chronic, mild, or undiagnosed that causes post-traumatic osteoarthritis. In another aspect, the patient did not receive a concurrent antibiotic or a probiotic therapy. In another aspect, the method further comprises eliminating one or more of the bacterial selected from Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae. In another aspect, the method further comprises providing the human patient a bacterial composition that is modified to correct a flora that will lead to osteoarthritis to a normal flora is provided before, during, or after undergoing a colon cleanse treatment, delivered by colonoscope, wherein the bacterial composition is not a fecal transplant, or wherein bacteria are lab-grown, or wherein bacteria are lab-grown and customized to specifically modify the flora of a specific patient for preventing, reducing or eliminating progression of osteoarthritis.

As embodied and broadly described herein, an aspect of the present disclosure relates to a composition for preventing, reducing or eliminating progression of osteoarthritis in a human patient comprising, consisting essentially of, or consisting of: one or more probiotic bacteria selected from at least one of: Actinobacteria actinomycetales; Actinobacteria coriobacteriia; Coriobacteriia coriobacteriales; Bacilli bacillales; Bacilli lactobacillales Clostridia clostridiales, in an effective amount sufficient to prevent, reduce, or eliminate the progression of osteoarthritis. In another aspect, the composition is a fecal transplant, an amino acid mixture, or both to promotes growth of at least one of: Actinobacteria actinomycetales; Actinobacteria coriobacteriia; Coriobacteriia coriobacteriales; Bacilli bacillales; Bacilli lactobacillales Clostridia clostridiales; or an agent that eliminates one or more of the bacterial selected from Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae; or both.

As embodied and broadly described herein, an aspect of the present disclosure relates to an assay to determine a biological profile associated with a bacterial flora that exacerbates progression of osteoarthritis from a fecal sample from a patient that compares the biological profile from the patient to a database of a normal biological profile that does not have progression of osteoarthritis, comprising, consisting essentially of, or consisting of: wherein the bacterial flora detected is selected from at least one of: Actinobacteria actinomycetales; Actinobacteria coriobacteriia; Coriobacteriia coriobacteriales; Bacilli bacillales; Bacilli lactobacillales Clostridia clostridiales; Actinobacteria actinomycetales; Actinobacteria coriobacteriia; Coriobacteriia coriobacteriales; Bacilli bacillales; Bacilli lactobacillales Clostridia clostridiales; Bacteroidetes and Firmicutes clostridia bacteria in an intestinal flora of the human patient. In another aspect, the biological profile is further defined as comprising an array that specifically tests for an amino acid profile associated with a presence, absence, or amount of the at least one of: Actinobacteria actinomycetales; Actinobacteria coriobacteriia; Coriobacteriia coriobacteriales; Bacilli bacillales; Bacilli lactobacillales Clostridia clostridiales; Actinobacteria actinomycetales; Actinobacteria coriobacteriia; Coriobacteriia coriobacteriales; Bacilli bacillales; Bacilli lactobacillales Clostridia clostridiales; Bacteroidetes and Firmicutes clostridia bacteria; or wherein the biological profile is further defined as comprising an array that specifically tests for a nucleic acid profile associated with a presence, absence, or amount of the at least one of: Actinobacteria actinomycetales; Actinobacteria coriobacteriia; Coriobacteriia coriobacteriales; Bacilli bacillales; Bacilli lactobacillales Clostridia clostridiales; Actinobacteria actinomycetales; Actinobacteria coriobacteriia; Coriobacteriia coriobacteriales; Bacilli bacillales; Bacilli lactobacillales Clostridia clostridiales; Bacteroidetes and Firmicutes clostridia.

As embodied and broadly described herein, an aspect of the present disclosure relates to a kit for screening a patient for progression of osteoarthritis and choosing a treatment based on the screening, the kit comprising, consisting essentially of, or consisting of: a screening tool that screens the patient for a risk of progression of osteoarthritis; a fecal sample collection container for determining a bacterial flora in a fecal sample of the patient; instructions for implementing a treatment to prevent, reduce, or eliminate the progression of osteoarthritis; and a treatment composition comprising one or more probiotic bacteria selected from at least one of: Actinobacteria actinomycetales; Actinobacteria coriobacteriia; Coriobacteriia coriobacteriales; Bacilli bacillales; Bacilli lactobacillales Clostridia clostridiales bacteria in an intestinal flora of the patient, wherein the composition comprises an amount effective to prevent, reduce, or eliminate the progression of osteoarthritis. In another aspect, the bacterial flora detected is a presence or an amount of the at least one of: Actinobacteria actinomycetales; Actinobacteria coriobacteriia; Coriobacteriia coriobacteriales; Bacilli bacillales; Bacilli lactobacillales Clostridia clostridiales bacteria in a fecal sample. In another aspect, the kit comprises a container with reagents for detecting at a genus or species level the bacterial flora selected from at least one of Ion Torrent Personal Genome Machine (PGM), next-generation sequencing (NGS), or qualitative Polymerase Chain Reaction (qPCR).

As embodied and broadly described herein, an aspect of the present disclosure relates to a method of diagnosing and treating a patient for progression of osteoarthritis, the method comprising, consisting essentially of, or consisting of: using an algorithm implemented in a computer program to: screen a patient for progression of osteoarthritis risk by scoring a questionnaire based on answers from the patient to the questionnaire, the scoring providing a cumulative point total, and determining the progression of osteoarthritis of the patient based on the cumulative point total from the questionnaire; obtaining or having obtained a biological sample from the patient to determine the presence, absence, or amount of at least 1, 2, 3, 4, 5, 6, 7, 8, 9, 10, 11, 12, 13, 14, 15, 16, or 17 bacteria selected from: Actinobacteria actinomycetales; Actinobacteria coriobacteriia; Coriobacteriia coriobacteriales; Coriobacteriia Coriobacteriales coriobacteriaceae; Coriobacteriia coriobacteriales coriobacteriaceae adlercreutzia; Bacilli bacillales; Bacilli bacillales staphylococcaceae; Bacilli bacillales staphylococcaceae Staphylococcus; Bacilli lactobacillales aerococcaceae; Clostridia clostridiales dehalobacteriaceae; Clostridia clostridiales dehalobacteriaceae dehalobacterium; Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae in the biological sample; and choosing a progression of osteoarthritis treatment if the progression of osteoarthritis risk exceeds a threshold, wherein the treatment comprises: a composition comprising at least one of: Actinobacteria actinomycetales; Actinobacteria coriobacteriia; Coriobacteriia coriobacteriales; Bacilli bacillales; Bacilli lactobacillales Clostridia clostridiales; one or more agents that increase an amount of the Actinobacteria actinomycetales; Actinobacteria coriobacteriia; Coriobacteriia coriobacteriales; Bacilli bacillales; Bacilli lactobacillales Clostridia clostridiales in the intestinal flora of the human patient; or one or more agents that decrease the amount of Bacteroidetes and Firmicutes clostridia bacteria in an intestinal flora of the human patient, bacteria to the patient, wherein the composition or transplant comprises an amount effective of the bacteria to prevent, reduce, or eliminate the progression of osteoarthritis. In another aspect, the patient preparation instructions include following dietary modifications in accordance with the progression of osteoarthritis treatment to at least one of: increase the amount of at least one of: Actinobacteria actinomycetales; Actinobacteria coriobacteriia; Coriobacteriia coriobacteriales; Bacilli bacillales; Bacilli lactobacillales Clostridia clostridiales bacteria in the intestinal flora of the patient as being different from the patient with the progression of osteoarthritis when compared to a normal subject without osteoarthritis.

A person of skill in the art would readily recognize that steps of various above-described methods can be performed by programmed computers. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Lleal M, Sarrabayrouse G, Willamil J, Santiago A, Pozuelo M, Manichanh C. A single fecal microbiota transplantation modulates the microbiome and improves clinical manifestations in a rat model of colitis. EBioMedicine 2019. Available at: http://dx.doi.org/10.1016/j.ebiom.2019.10.002.
2. Manichanh C, Reeder J, Gibert P, Varela E, Llopis M, Antolin M, et al. Reshaping the gut microbiome with bacterial transplantation and antibiotic intake. Genome Res 2010; 20:1411-1419.
3. Glasson S S, Blanchet T J, Morris E A. The surgical destabilization of the medial meniscus (DMM) model of osteoarthritis in the 129/SvEv mouse. Osteoarthritis Cartilage 2007; 15:1061-1069.
4. Glasson S S, Chambers M G, Van Den Berg W B, Little C B. The OARSI histopathology initiative—recommendations for histological assessments of osteoarthritis in the mouse. Osteoarthritis and Cartilage 2010; 18:S17-523. Available at: http://dx.doi.org/10.1016/j.joca.2010.05.025.
5. Anon. 16S Sample Preparation Guide. Available at: https://support.illumina.com/documents/documentation/chemistry_documentation/16s/16s-metagenomic-library-prep-guide-15044223-b.pdf.
6. Caporaso J G, Kuczynski J, Stombaugh J, Bittinger K, Bushman F D, Costello E K, et al. QIIME allows analysis of high-throughput community sequencing data. Nat Methods 2010; 7:335-336.
7. Edgar RC. Search and clustering orders of magnitude faster than BLAST. Bioinformatics 2010; 26:2460-2461.
8. McDonald D, Price M N, Goodrich J, Nawrocki E P, DeSantis T Z, Probst A, et al. An improved Greengenes taxonomy with explicit ranks for ecological and evolutionary analyses of bacteria and archaea. ISME J 2012; 6:610-618.
9. Chang Q, Luan Y, Sun F. Variance adjusted weighted UniFrac: a powerful beta diversity measure for comparing communities based on phylogeny. BMC Bioinformatics 2011; 12:118.
10. Hamady M, Knight R. Microbial community profiling for human microbiome projects: Tools, techniques, and challenges. Genome Res 2009; 19:1141-1152.
11. Segata N, Izard J, Waldron L, Gevers D, Miropolsky L, Garrett W S, et al. Metagenomic biomarker discovery and explanation. Genome Biol 2011; 12:R60.
12. Kruskal W H, Wallis W A. Use of Ranks in One-Criterion Variance Analysis. J Am Stat Assoc 1952; 47:583-621.
13. Fisher R A. THE USE OF MULTIPLE MEASUREMENTS IN TAXONOMIC PROBLEMS. Ann Eugen 1936; 7:179-188.
14. Battaglia T. LEfSe An Introduction to QIIME 1.9.1. Available at: https://twbattaglia.gitbooks.io/introduction-to-qiime/content/lefse.html. Accessed Feb. 14, 2018.
15. Langille M G I, Zaneveld J, Caporaso J G, McDonald D, Knights D, Reyes J A, et al. Predictive functional profiling of microbial communities using 16S rRNA marker gene sequences. Nat Biotechnol 2013; 31:814-821.
16. Kanehisa M, Goto S. KEGG: kyoto encyclopedia of genes and genomes. Nucleic Acids Res 2000; 28:27-30.
17. Parks D H, Tyson G W, Hugenholtz P, Beiko R G. STAMP: statistical analysis of taxonomic and functional profiles. Bioinformatics 2014; 30:3123-3124.

What is claimed is:

1. A method of preventing, reducing, or eliminating progression of osteoarthritis in a human patient comprising:
identifying a human patient in need of treatment for progression of osteoarthritis associated with an altered intestinal flora; and
providing the human patient with at least one of:
a composition comprising at least one of: Actinobacteria actinomycetales; Actinobacteria coriobacteriia; Coriobacteriia coriobacteriales; Bacilli bacillales; Bacilli lactobacillales Clostridia clostridiales;
one or more agents that increase an amount of the Actinobacteria actinomycetales; Actinobacteria coriobacteriia; Coriobacteriia coriobacteriales; Bacilli bacillales; Bacilli lactobacillales Clostridia clostridiales in the intestinal flora of the human patient; or
one or more agents that decrease the amount of Bacteroidetes and Firmicutes clostridia bacteria in an intestinal flora of the human patient,
wherein the composition at least one of prevents, reduces or eliminates progression of osteoarthritis.

2. The method of claim 1, wherein the human patient has early symptomatic osteoarthritis or a traumatic injury.

3. The method of claim 1, wherein the composition further comprises an amino acid mixture that promotes growth of the at least one of: Actinobacteria actinomycetales; Actinobacteria coriobacteriia; Coriobacteriia coriobacteriales; Bacilli bacillales; Bacilli lactobacillales Clostridia clostridiales bacteria; or a transplantation with the at least one of: Actinobacteria actinomycetales; Actinobacteria coriobacteriia; Coriobacteriia coriobacteriales; Bacilli bacillales; Bacilli lactobacillales Clostridia clostridiales increases microbiome-mediated cartilage regeneration.

4. The method of claim 1, wherein the human patient does not receive a concurrent antibiotic or a probiotic therapy.

5. The method of claim 1, further comprising providing the human patient one or more bacteria selected from Actinobacteria actinomycetales; Actinobacteria coriobacteriia; Coriobacteriia coriobacteriales; Coriobacteriia Coriobacteriales coriobacteriaceae; Coriobacteriia coriobacteriales coriobacteriaceae adlercreutzia; Bacilli bacillales; Bacilli bacillales staphylococcaceae; Bacilli bacillales staphylococcaceae Staphylococcus; Bacilli lactobacillales aerococcaceae; Clostridia clostridiales dehalobacteriaceae; Clostridia clostridiales dehalobacteriaceae dehalobacterium.

6. The method of claim 1, further comprising eliminating one or more of the bacterial selected from Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae.

7. The method of claim 1, further comprising providing the human patient a bacterial composition that is modified to prevent, reduce or eliminate progression of osteoarthritis before, during, or after undergoing a colon cleanse treatment, delivered by colonoscope, wherein the bacterial composition is not a fecal transplant, or wherein bacteria are lab-grown, or wherein bacteria are lab-grown and customized to specifically modify a flora of a specific patient to prevent, reduce or eliminate progression of osteoarthritis.

8. A method of identifying a patient in need of preventing, reducing or eliminating progression of osteoarthritis that will benefit from a probiotic bacteria treatment comprising:
identifying a human patient with progression of osteoarthritis;
obtaining a biological sample from the patient that comprises gut intestinal flora; and
determining whether the gut intestinal flora in the biological sample comprises a decrease in a presence of probiotic bacteria selected from at least one of: Actinobacteria actinomycetales; Actinobacteria coriobacteriia; Coriobacteriia coriobacteriales; Coriobacteriia Coriobacteriales coriobacteriaceae; Coriobacteriia coriobacteriales coriobacteriaceae adlercreutzia; Bacilli bacillales; Bacilli bacillales staphylococcaceae; Bacilli bacillales staphylococcaceae Staphylococcus; Bacilli lactobacillales aerococcaceae; Clostridia clostridiales dehalobacteriaceae; Clostridia clostridiales dehalobacteriaceae dehalobacterium; Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae, when compared to a human sample that does not have osteoarthritis.

9. The method of claim 8, further comprising treating the patient with a composition comprising one or more probiotic bacteria selected from Actinobacteria actinomycetales; Actinobacteria coriobacteriia; Coriobacteriia coriobacteriales; Bacilli bacillales; Bacilli lactobacillales Clostridia clostridiales, wherein a composition comprises an amount effective to reduce or eliminate the progression of osteoarthritis; or a fecal transplant comprising at least one of: Actinobacteria actinomycetales; Actinobacteria coriobacteriia; Coriobacteriia coriobacteriales; Bacilli bacillales; Bacilli lactobacillales Clostridia clostridiales, or an amino acid mixture that increases the Actinobacteria actinomycetales; Actinobacteria coriobacteriia; Coriobacteriia coriobacteriales; Bacilli bacillales; Bacilli lactobacillales Clostridia clostridiales.

10. The method of claim 8, wherein a presence of a probiotic bacteria is determined by at least one of metagenomic shotgun sequencing of bacteria, quantitative PCR assays or metabolic LC-MS analysis of bacterial metabolites.

11. The method of claim 8, wherein the patient has an injury that is chronic, mild, or undiagnosed that causes post-traumatic osteoarthritis.

12. The method of claim 8, wherein the patient did not receive a concurrent antibiotic or a probiotic therapy.

13. The method of claim 8, further comprising eliminating one or more of the bacterial selected from Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae.

14. The method of claim 8, further comprising providing the patient a bacterial composition that is modified to correct a flora that will lead to osteoarthritis to a normal flora is provided before, during, or after undergoing a colon cleanse treatment, delivered by colonoscope, wherein the bacterial composition is not a fecal transplant, or wherein bacteria are lab-grown, or wherein bacteria are lab-grown and customized to specifically modify the flora of a specific patient for preventing, reducing or eliminating progression of osteoarthritis.

15. A composition for preventing, reducing or eliminating progression of osteoarthritis in a human patient comprising one or more probiotic bacteria selected from at least one of: Actinobacteria actinomycetales; Actinobacteria coriobacteriia; Coriobacteriia coriobacteriales; Bacilli bacillales; Bacilli lactobacillales Clostridia clostridiales, in an effective amount sufficient to prevent, reduce, or eliminate the progression of osteoarthritis.

16. The composition of claim 15, wherein the composition is a fecal transplant, an amino acid mixture, or both to promotes growth of at least one of: Actinobacteria actinomycetales; Actinobacteria coriobacteriia; Coriobacteriia coriobacteriales; Bacilli bacillales; Bacilli lactobacillales Clostridia clostridiales; or an agent that eliminates one or more of the bacterial selected from Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae; or both.

17. An assay to determine a biological profile associated with a bacterial flora that exacerbates progression of osteoarthritis from a fecal sample from a patient that compares the biological profile from the patient to a database of a normal biological profile that does not have progression of osteoarthritis, wherein the bacterial flora detected is selected from at least one of: Actinobacteria actinomycetales; Actinobacteria coriobacteriia; Coriobacteriia coriobacteriales; Bacilli bacillales; Bacilli lactobacillales Clostridia clostridiales; Actinobacteria actinomycetales; Actinobacteria coriobacteriia; Coriobacteriia coriobacteriales; Bacilli bacillales; Bacilli lactobacillales Clostridia clostridiales; Bacteroidetes and Firmicutes clostridia bacteria in an intestinal flora of the human patient.

18. The assay of claim 17, wherein the biological profile is further defined as comprising an array that specifically tests for an amino acid profile associated with a presence, absence, or amount of the at least one of: Actinobacteria actinomycetales; Actinobacteria coriobacteriia; Coriobacteriia coriobacteriales; Bacilli bacillales; Bacilli lactobacillales Clostridia clostridiales; Actinobacteria actinomycetales; Actinobacteria coriobacteriia; Coriobacteriia coriobacteriales; Bacilli bacillales; Bacilli lactobacillales Clostridia clostridiales; Bacteroidetes and Firmicutes clostridia bacteria; or wherein the biological profile is further defined as comprising an array that specifically tests for a nucleic acid profile associated with a presence, absence, or amount of the at least one of: Actinobacteria actinomycetales; Actinobacteria coriobacteriia; Coriobacteriia coriobacteriales; Bacilli bacillales; Bacilli lactobacillales Clostridia clostridiales; Actinobacteria actinomycetales; Actinobacteria coriobacteriia; Coriobacteriia coriobacteriales; Bacilli bacillales; Bacilli lactobacillales Clostridia clostridiales; Bacteroidetes and Firmicutes clostridia.

19. A kit for screening a patient for progression of osteoarthritis and choosing a treatment based on the screening, the kit comprising:
a screening tool that screens the patient for a risk of progression of osteoarthritis;
a fecal sample collection container for determining a bacterial flora in a fecal sample of the patient;
instructions for implementing a treatment to prevent, reduce, or eliminate the progression of osteoarthritis; and
a treatment composition comprising one or more probiotic bacteria selected from at least one of: Actinobacteria actinomycetales; Actinobacteria coriobacteriia; Coriobacteriia coriobacteriales; Bacilli bacillales; Bacilli lactobacillales Clostridia clostridiales bacteria in an intestinal flora of the patient, wherein the composition comprises an amount effective to prevent, reduce, or eliminate the progression of osteoarthritis.

20. The kit of claim 19, wherein the bacterial flora detected is a presence or an amount of the at least one of: Actinobacteria actinomycetales; Actinobacteria coriobacteriia; Coriobacteriia coriobacteriales; Bacilli bacillales; Bacilli lactobacillales Clostridia clostridiales bacteria in a fecal sample.

21. The kit of claim 19, wherein the kit comprises a container with reagents for detecting at a genus or species level the bacterial flora selected from at least one of Ion Torrent Personal Genome Machine (PGM), next-generation sequencing (NGS), or qualitative Polymerase Chain Reaction (qPCR).

22. A method of diagnosing and treating a patient for progression of osteoarthritis, the method comprising:
using an algorithm implemented in a computer program to: screen a patient for progression of osteoarthritis risk by scoring a questionnaire based on answers from the patient to the questionnaire, the scoring providing a cumulative point total, and determining the progression of osteoarthritis of the patient based on the cumulative point total from the questionnaire;
obtaining or having obtained a biological sample from the patient to determine the presence, absence, or amount of at least 1, 2, 3, 4, 5, 6, 7, 8, 9, 10, 11, 12, 13, 14, 15, 16, or 17 bacteria selected from: Actinobacteria actinomycetales; Actinobacteria coriobacteriia; Coriobacteriia coriobacteriales; Coriobacteriia Coriobacteriales coriobacteriaceae; Coriobacteriia coriobacteriales coriobacteriaceae adlercreutzia; Bacilli bacillales; Bacilli bacillales staphylococcaceae; Bacilli bacillales staphylococcaceae Staphylococcus; Bacilli lactobacillales aerococcaceae; Clostridia clostridiales dehalobacteriaceae; Clostridia clostridiales dehalobacteriaceae dehalobacterium; Bacteroidetes; Bacteroidetes bacteroidia; Bacteroidetes bacteroidia bacteroidales; Bacteroidetes bacteroidia bacteroidales rikenellaceae; Firmicutes clostridia clostridiales christensenellaceae; Firmicutes clostridia clostridiales lachnospiraceae anaerostipes; Firmicutes clostridia clostridiales ruminococcaceae in the biological sample; and
choosing a progression of osteoarthritis treatment if the progression of osteoarthritis risk exceeds a threshold, wherein the treatment comprises:
a composition comprising at least one of:
Actinobacteria actinomycetales; Actinobacteria coriobacteriia; Coriobacteriia coriobacteriales; Bacilli bacillales; Bacilli lactobacillales Clostridia clostridiales;
one or more agents that increase an amount of the Actinobacteria actinomycetales; Actinobacteria coriobacteriia; Coriobacteriia coriobacteriales; Bacilli bacillales; Bacilli lactobacillales Clostridia clostridiales in the intestinal flora of the human patient; or
one or more agents that decrease the amount of Bacteroidetes and Firmicutes clostridia bacteria in an intestinal flora of the human patient, bacteria to the patient, wherein the composition or transplant comprises an amount effective of the bacteria to prevent, reduce, or eliminate the progression of osteoarthritis.

23. The method of claim 22, wherein patient preparation instructions include following dietary modifications in accordance with the progression of osteoarthritis treatment to at least one of: increase the amount of at least one of: Actinobacteria actinomycetales; Actinobacteria coriobacteriia; Coriobacteriia coriobacteriales; Bacilli bacillales; Bacilli lactobacillales Clostridia clostridiales bacteria in the intestinal flora of the patient as being different from the patient with the progression of osteoarthritis when compared to a normal subject without osteoarthritis.
